(19) United States
(12) Patent Application Publication
(10) Pub. No.: US 2014/0194752 A1
(43) Pub. Date: Jul. 10, 2014

(54) BIO-OPTICAL MEASUREMENT APPARATUS AND MEASUREMENT PROBE

(71) Applicant: OLYMPUS MEDICAL SYSTEMS CORP., Tokyo (JP)

(72) Inventor: Kenji KAMIMURA, Hachioji-shi (JP)

(21) Appl. No.: 14/206,338

(22) Filed: Mar. 12, 2014

Related U.S. Application Data
(63) Continuation of application No. PCT/JP2012/082941, filed on Dec. 19, 2012.
(60) Provisional application No. 61/597,326, filed on Feb. 10, 2012.

(51) Int. Cl. A61B 5/00

(52) U.S. Cl. CPC A61B 5/0062
USPC 600/476

(57) ABSTRACT

A measurement probe is detachably connected to a bio-optical measurement apparatus which performs optical measurement on body tissue. The measurement probe includes two illumination fibers configured to irradiate the body tissue with low coherent light having a short spatial coherence length, and three or more light-receiving fibers configured to receive scattered light at multiple angles. The three or more light-receiving fibers are arranged on a perpendicular bisector of a line segment connecting centers of the two illumination fibers at a distal end surface of the measurement probe.

### CROSS-REFERENCE TO RELATED APPLICATIONS

This application is a continuation of PCT international application Ser. No. PCT/JP2012/082941 filed on Dec. 19, 2012 which designates the United States, incorporated herein by reference, and which claims the benefit of priority from U.S. provisional application No. 61/597,326, filed on Feb. 10, 2012, incorporated herein by reference.

### BACKGROUND OF THE INVENTION

1. Field of the Invention

The present invention relates to a bio-optical measurement apparatus that measures optical properties of scattered light and to a measurement probe for measurement.

2. Description of the Related Art

In recent years, there has been a known bio-optical measurement apparatus that irradiates body tissue with illumination light and that estimates the properties of the body tissue on the basis of a measurement value of detected light that is reflected or scattered by the body tissue. Such a bio-optical measurement apparatus is used in combination with an endoscope that observes an organ, such as a digestive organ. For example, there is a proposed bio-optical measurement apparatus that uses low-coherence enhanced backscattering (LEBS) technology that detects the properties of body tissue by irradiating the body tissue with white light that is low coherent light having a short spatial coherence length, from the distal end of an illumination fiber of a measurement probe and measuring, using multiple light-receiving fibers, the intensity distribution of scattered light at multiple angles (see International Publication Pamphlet No. WO 2007/133684).

### SUMMARY OF THE INVENTION

In some embodiments, a measurement probe is detachably connected to a bio-optical measurement apparatus which performs optical measurement on body tissue. The measurement probe includes two illumination fibers configured to irradiate the body tissue with low coherent light having a short spatial coherence length, and three or more light-receiving fibers configured to receive scattered light at multiple angles. The three or more light-receiving fibers are arranged on a perpendicular bisector of a line segment connecting centers of the two illumination fibers at a distal end surface of the measurement probe.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram schematically illustrating the configuration of a bio-optical measurement apparatus according to a first embodiment of the present invention;

FIG. 2 is a sectional view illustrating a distal end portion, cut in the longitudinal direction, of a measurement probe illustrated in FIG. 1;

FIG. 3 is a schematic diagram illustrating the distal end surface of the measurement probe illustrated in FIG. 2;

FIG. 4 is a schematic diagram illustrating a distal end surface of a measurement probe in a bio-optical measurement apparatus according to a modification of the first embodiment of the present invention;

FIG. 5 is a schematic diagram illustrating a distal end surface of a measurement probe in a bio-optical measurement apparatus according to a second embodiment of the present invention;

FIG. 6 is a sectional view illustrating a distal end portion, cut in the longitudinal direction, of a measurement probe in a bio-optical measurement apparatus according to a third embodiment of the present invention;

FIG. 7 is a sectional view illustrating the distal end surface of the measurement probe in the bio-optical measurement apparatus according to the third embodiment of the present invention; and

FIG. 8 is a sectional view illustrating a distal end surface of a measurement probe in a bio-optical measurement apparatus according to a fourth embodiment of the present invention.

### DETAILED DESCRIPTION OF THE PREFERRED EMBODIMENTS

In the following, preferred embodiments of a bio-optical measurement apparatus and a measurement probe according to the present invention will be described in detail with reference to the accompanying drawings. The present invention is not limited to the embodiments. In the drawings, components that are identical to those in embodiments are assigned the same reference numerals. The drawings used for the descriptions below are only schematic illustrations. The relationship between the thickness and the width of each member, the proportions of each member, and so on are different from those used in practice. The size or reduction in scale of elements may sometimes differ between the drawings.

### First Embodiment

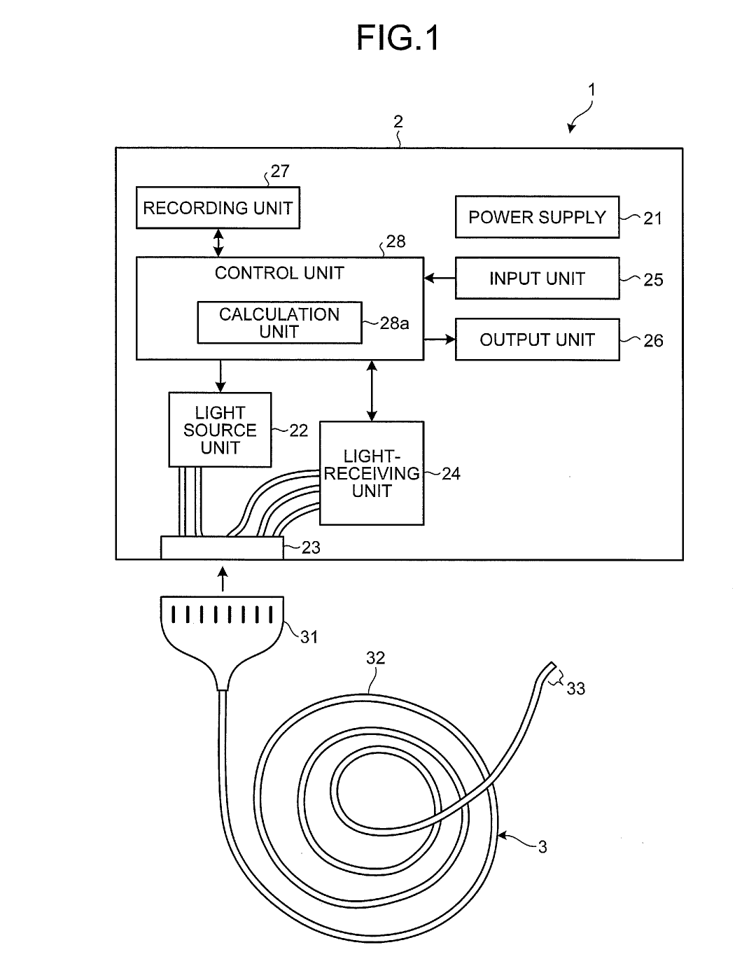

FIG. 1 is a block diagram schematically illustrating the configuration of a bio-optical measurement apparatus according to a first embodiment of the present invention. As illustrated in FIG. 1, a bio-optical measurement apparatus 1 includes a main body unit 2 that performs optical measurement on a measurement object, such as body tissue that is a scatterer, and that detects the properties of a measurement object and, furthermore, the bio-optical measurement apparatus 1 includes a measurement probe 3 that is used for measurement and is inserted into a subject.

The main body unit 2 includes a power supply 21, a light source unit 22, a connecting unit 23, a light-receiving unit 24, an input unit 25, an output unit 26, a recording unit 27, and a control unit 28. The power supply 21 supplies electrical power to each component of the main body unit 2.

The light source unit 22 is implemented by using an incoherent light source, such as a white light emitting diode (LED), a xenon lamp, a tungsten lamp, or a halogen lamp, and by using, as necessary, a single lens or multiple lenses. The light source unit 22 supplies, to the measurement probe 3 via the connecting unit 23, incoherent light that has at least one spectral component that irradiates the measurement object with light.

The connecting unit 23 detachably connects the proximal end of the measurement probe 3 to the main body unit 2. The connecting unit 23 supplies light emitted from the light source unit 22 to the measurement probe 3 and outputs, to the light-receiving unit 24, scattered light that is output from the measurement probe 3. The connecting unit 23 outputs, to the control unit 28, information on the connection status of the measurement probe 3.

The light-receiving unit 24 receives scattered light, which is emitted from the measurement probe 3 and is scattered on the measurement object, and measures the scattered light. The light-receiving unit 24 is implemented by using multiple spectrometers. Specifically, the spectrometer in the light-receiving unit 24 is arranged in accordance with the number of light-receiving fibers of the measurement probe 3, which will be described later. The light-receiving unit 24 measures spectral components and intensity distribution of the scattered light incident from the measurement probe 3 and measures each wavelength. The light-receiving unit 24 outputs the measurement results to the control unit 28.

The input unit 25 is implemented by using, for example, a push button or touch panel switch; receives a start signal that instructs the bio-optical measurement apparatus 1 to start or an operation signal that instructs to operate various operations; and outputs the signals to the control unit 28.

The output unit 26 is implemented by using a display, such as a liquid crystal display or an organic electro luminescence (EL) display, and by using a speaker and outputs information on the various processes performed by the bio-optical measurement apparatus 1.

The recording unit 27 is implemented by using a volatile memory or a nonvolatile memory and records various programs used to operate the bio-optical measurement apparatus 1 and various kinds of data or parameters used for an optical measurement process. The recording unit 27 temporarily records information that is being processed by the bio-optical measurement apparatus 1. Furthermore, the recording unit 27 records the measurement results obtained by the bio-optical measurement apparatus 1. The recording unit 27 may also be configured by using a memory card mounted outside the bio-optical measurement apparatus 1.

The control unit 28 is configured by a central processing unit (CPU) or the like. The control unit 28 controls the flow of processes performed by each unit in the bio-optical measurement apparatus 1. The control unit 28 controls the operation of the bio-optical measurement apparatus 1 by transferring instruction information or data with respect to each unit in the bio-optical measurement apparatus 1. The control unit 28 records, in the recording unit 27, the measurement results obtained by the light-receiving unit 24. The control unit 28 includes a calculation unit 28a.

The calculation unit 28a performs multiple calculation processes on the basis of the measurement results obtained by the light-receiving unit 24 and calculates characteristic values related to the properties of the measurement object. The type of the characteristic values is set in accordance with, for example, an instruction signal received by the input unit 25.

The measurement probe 3 is implemented by using multiple optical fibers. Specifically, the measurement probe 3 is implemented by using illumination fibers that emit illumination light to the measurement object and by using multiple light-receiving fibers in which reflected light reflected by the measurement object and/or scattered light is incident at different angles. The measurement probe 3 includes a proximal end portion 31 that is detachably connected to the connecting unit 23 in the main body unit 2; a flexible portion 32 that has flexibility; and a distal end portion 33 through which light supplied from the light source unit 22 is emitted and reflected light reflected by the measurement object and/or scattered light enters.

The bio-optical measurement apparatus 1 having the above-described configuration is inserted into a subject via the treatment instrument channel arranged in the endoscope apparatus in the endoscope system; irradiates the measurement object with illumination light; and receives, by the light-receiving unit 24, the reflected light reflected by the measurement object and/or scattered light. Then, the calculation unit 28a measures, on the basis of the received light results output from the light-receiving unit 24, the properties of the measurement object.

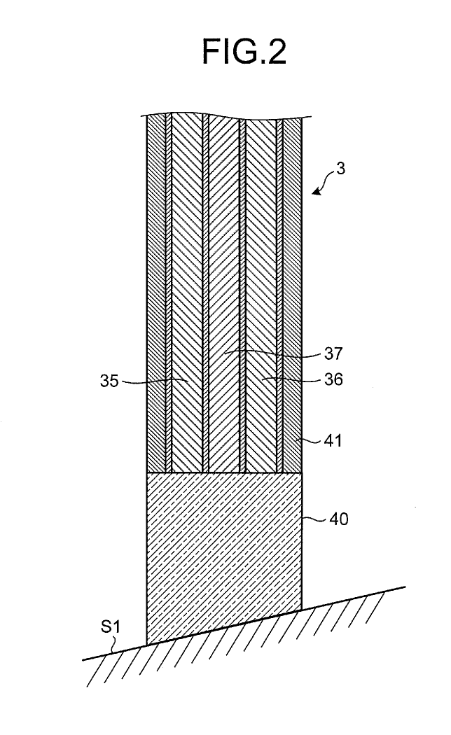

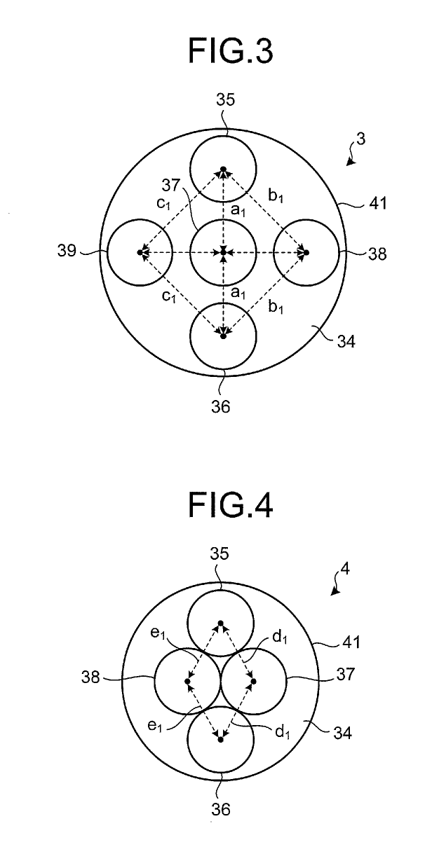

In the following, the measurement probe 3 illustrated in FIG. 1 will be described in detail. FIG. 2 is a sectional view illustrating a distal end portion, cut in the longitudinal direction, of the measurement probe 3 illustrated in FIG. 1. FIG. 3 is a schematic diagram illustrating a distal end surface 34 of the measurement probe illustrated in FIG. 2.

As illustrated in FIGS. 2 and 3, the measurement probe 3 includes a first illumination fiber 35, a second illumination fiber 36, a first light-receiving fiber 37, a second light-receiving fiber 38, a third light-receiving fiber 39, and an optical member 40. The sides of the first illumination fiber 35, the second illumination fiber 36, the first light-receiving fiber 37, the second light-receiving fiber 38, and the third light-receiving fiber 39 are covered by a covering layer 41 to block light and prevent damage.

The first illumination fiber 35 and the second illumination fiber 36 propagate illumination light supplied from the light source unit 22 to the distal end and irradiates measurement object S1 with light from the distal end.

Each of the first light-receiving fiber 37, the second light-receiving fiber 38, and the third light-receiving fiber 39 outputs, from the proximal end, reflected light that enters from each of the distal ends and that is reflected by the measurement object S1 and/or scattered light.

The optical member 40 fixes the distance between the first illumination fiber 35 and the measurement object S1 and the distance between the second illumination fiber 36 and the measurement object S1 such that light is irradiated in a state in which the spatial coherence length is reliably kept constant. Furthermore, the optical member 40 fixes the distance between the first light-receiving fiber 37 and the measurement object S1, the distance between the second light-receiving fiber 38 and the measurement object S1, and the distance between the third light-receiving fiber 39 and the measurement object S1 such that light having a predetermined scattering angle is stably received. Furthermore, because the surface of the measurement object S1 is flattened by the bottom surface of the optical member 40, measurements can be performed without being affected by the irregularities of the surface of the measurement object S1.

As illustrated in FIG. 3, in the measurement probe 3, the distances between any one of the light-receiving fibers and each of the illumination fibers are the same. Specifically, the first light-receiving fiber 37, the second light-receiving fiber 38, and the third light-receiving fiber 39 are arranged on the perpendicular bisector of the line connecting the center of the first illumination fiber 35 and the center of the second illumination fiber 36. For example, if the first light-receiving fiber 37 is taken as a reference, the distances to the first illumination fiber 35 and to the second illumination fiber 36 are the distance a₁. Furthermore, if the second light-receiving fiber 38 is taken as a reference, the distances to the first illumination fiber 35 and to the second illumination fiber 36 are the distance b₁. Furthermore, if the third light-receiving fiber 39 is taken as a reference, the distances to the first illumination fiber 35 and to the second illumination fiber 36 are the distance c₁.

In this way, in the measurement probe 3, the first illumination fiber 35 and the second illumination fiber 36 are arranged at positions where the distances between any of the light-receiving fibers and each of the illumination fibers are the same. Accordingly, because each of the scattering angles of scattered light received by each of the first light-receiving fiber 37, the second light-receiving fiber 38, and the third light-receiving fiber 39 is uniquely fixed, it is possible to receive an angular component, which is the same component as that obtained when using a single illumination fiber, having the light level increased by a factor of 2. Accordingly, light having a sufficient intensity can be irradiated and the diameter of the measurement probe can be reduced.

According to the first embodiment of the present invention described above, in the measurement probe 3, if any one of the light-receiving fibers is taken as a reference, the first illumination fiber 35 and the second illumination fiber 36 are arranged such that the distances to each of the illumination fibers are the same. Accordingly, light having a sufficient intensity can be irradiated and the diameter of the measurement probe 3 can be reduced.

Furthermore, according to the first embodiment of the present invention, in the measurement probe 3, the first illumination fiber 35 and the second illumination fiber 36 are arranged such that the distances between any of the light-receiving fibers and each of the illumination fibers are the same. Accordingly, it is possible to receive, without light received by each of the light-receiving fibers being interfered with, light that has the same properties and has the light level increased by a factor of 2, and thereby the SN ratio can be improved.

In the first embodiment, three light-receiving fibers are arranged; however, as illustrated in FIG. 4, this embodiment can also be used in practice even if two light-receiving fibers are used. In such a case, in a measurement probe 4, the first illumination fiber 35 and the second illumination fiber 36 are arranged such that the distances (e₁, d₁) between any of the light-receiving fibers and each of the illumination fibers are the same. In such a case, the other light-receiving fiber may also be arranged on the perpendicular bisector of the line connecting the center of the first illumination fiber 35 and the center of the second illumination fiber 36. Furthermore, if a light-receiving fiber is arranged on the perpendicular bisector of the line connecting the center of the first illumination fiber 35 and the center of the second illumination fiber 36, it is possible to appropriately change the number of light-receiving fibers. Accordingly, light having a sufficient intensity can be irradiated and the diameter of the measurement probe 4 can be reduced.

### Second Embodiment

In the following, a second embodiment according to the present invention will be described. With a bio-optical measurement apparatus according to the second embodiment, the configuration of a measurement probe differs from that in the first embodiment described above. Accordingly, in the following, only the configuration of the measurement probe in the bio-optical measurement apparatus according to the second embodiment will be described. Components that are identical to those in embodiments are assigned the same reference numerals.

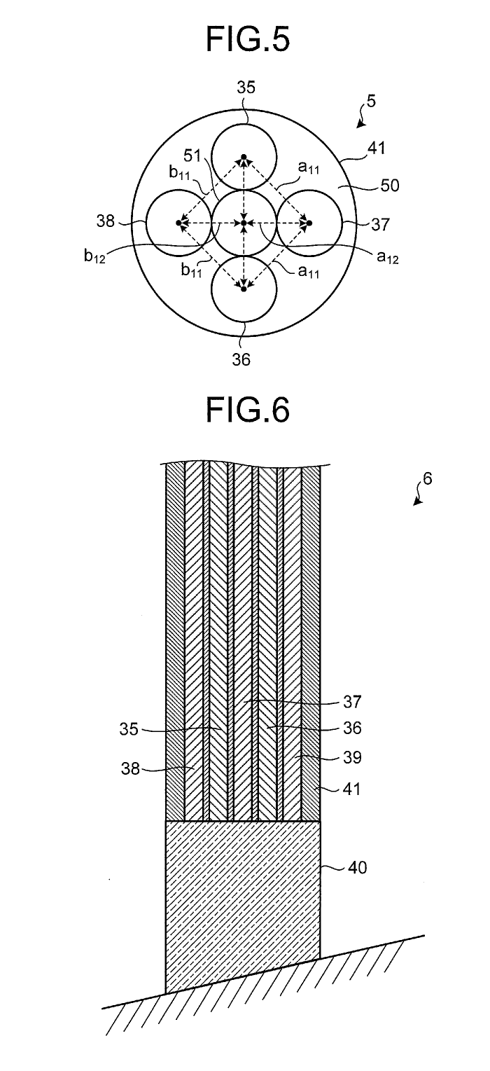

FIG. 5 is a schematic diagram illustrating a distal end surface 50 of a measurement probe 5 in the bio-optical measurement apparatus 1 according to a second embodiment of the present invention.

As illustrated in FIG. 5, the measurement probe 5 includes the first illumination fiber 35, the second illumination fiber 36, a third illumination fiber 51, the first light-receiving fiber 37, and the second light-receiving fiber 38. The third illumination fiber 51 has the same configuration as that of the first illumination fiber 35.

In the measurement probe 5, the distances between any of the light-receiving fibers and each of the illumination fibers are the same. Specifically, the first light-receiving fiber 37 and the second light-receiving fiber 38 are arranged on the perpendicular bisector of the line connecting the center of the first illumination fiber 35, the center of the second illumination fiber 36, and the third illumination fiber 51. For example, if the first light-receiving fiber 37 is taken as a reference, the distances to the first illumination fiber 35 and to the second illumination fiber 36 are the distance a₁₁ and the distance to the third illumination fiber 51 is a₁₂. Furthermore, if the second light-receiving fiber 38 is taken as a reference, the distances to the first illumination fiber 35 and to the second illumination fiber 36 are the distance b₁₁ and the distance to the third illumination fiber 51 is b₁₂.

In this way, in the measurement probe 5, the first illumination fiber 35, the second illumination fiber 36, and the third illumination fiber 51 are arranged such that the distances between any of the light-receiving fibers and each of the illumination fibers are substantially the same. Accordingly, because each of the scattering angles of scattered light received by each of the first light-receiving fiber 37 and the second light-receiving fiber 38 is uniquely fixed, it is possible to receive an angular component, which is the same component as that obtained when using a single illumination fiber, having the light level increased by a factor of 3. Accordingly, light having a sufficient intensity can be irradiated and the diameter of the measurement probe 5 can be reduced.

According to the second embodiment of the present invention described above, in the measurement probe 5, the first illumination fiber 35, the second illumination fiber 36, and the third illumination fiber 51 are arranged at positions where the distances between any of the light-receiving fibers and each of the illumination fibers are substantially the same. Accordingly, light having a sufficient intensity can be irradiated and the diameter of the measurement probe 5 can be reduced.

In the second embodiment, three illumination fibers are arranged; however, the number of illumination fibers can be appropriately changed as long as the alignment of the illumination fibers is orthogonal to that of the light-receiving fibers. Specifically, the number of illumination fibers can be appropriately changed as long as the illumination fibers are arranged in a straight line.

### Third Embodiment

In the following, a third embodiment according to the present invention will be described. With a bio-optical measurement apparatus according to the third embodiment, the configuration of a measurement probe differs from that in the embodiments described above. Accordingly, in the following, only the configuration of the measurement probe in the bio-optical measurement apparatus in the third embodiment will be described. Components that are identical to those in embodiments above are assigned the same reference numerals.

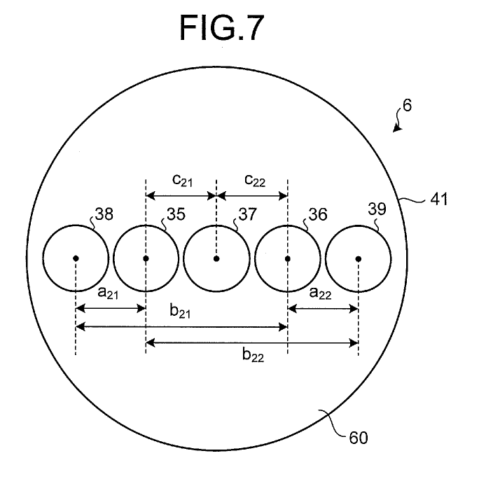

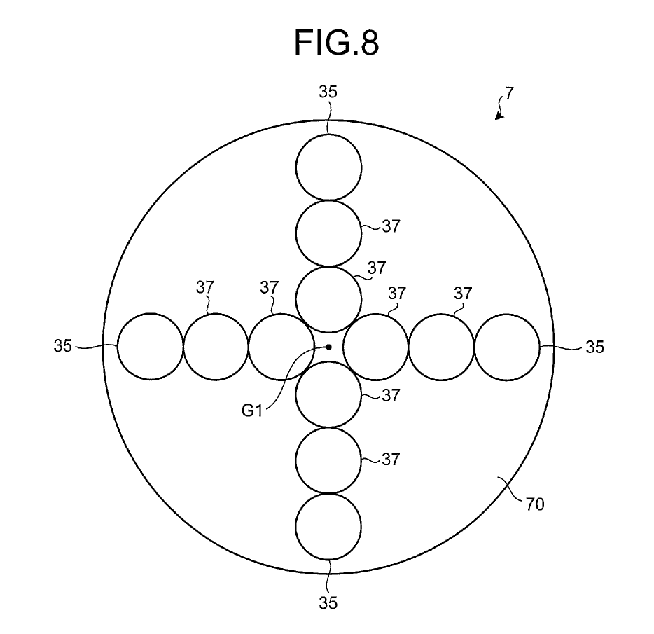

FIG. 6 is a sectional view illustrating a distal end portion, cut in the longitudinal direction, of a measurement probe 6 in the bio-optical measurement apparatus 1 according to a third embodiment of the present invention. FIG. 7 is a sectional view illustrating the distal end surface of the measurement probe 6 in the bio-optical measurement apparatus 1 according to the third embodiment of the present invention.

As illustrated in FIGS. 6 and 7, the measurement probe 6 includes the first illumination fiber 35, the second illumination fiber 36, the first light-receiving fiber 37, the second light-receiving fiber 38, the third light-receiving fiber 39, and the optical member 40. It is assumed that the sides of the first illumination fiber 35, the second illumination fiber 36, the first light-receiving fiber 37, the second light-receiving fiber 38, and the third light-receiving fiber 39 are covered by the covering layer 41 to block light and prevent damage. The distal end portions of the first illumination fiber 35, the second illumination fiber 36, the first light-receiving fiber 37, the second light-receiving fiber 38, and the third light-receiving fiber 39 are arranged in parallel each other. The first illumination fiber 35, the second illumination fiber 36, the first light-receiving fiber 37, the second light-receiving fiber 38, and the third light-receiving fiber 39 are alternately arranged in a straight line.

Furthermore, in the measurement probe 6, if a combination of light-receiving fibers is taken as a reference, the sums of the distances to each of the illumination fibers are equal. Specifically, if the second light-receiving fiber 38 is taken as a reference, the distance to the first illumination fiber 35 is the distance a₂₁ and the distance to the second illumination fiber 36 is the distance b₂₂. Furthermore, if the third light-receiving fiber 39 is taken as a reference, the distance to the first illumination fiber 35 is the distance b₂₂ and the distance to the second illumination fiber 36 is the distance a₂₂. Accordingly, if a combination of light-receiving fibers is taken as a reference, the sums of the distances to each of the illumination fibers are the same. Furthermore, if the first light-receiving fiber 37 is taken as a reference, the distance to the first illumination fiber 35 is the distance c₂₁ and the distance to the second illumination fiber 36 is the distance c₂₂.

In this way, in the measurement probe 6, if a combination of light-receiving fibers is taken as a reference, the first illumination fiber 35 and the second illumination fiber 36 are arranged at positions where the sums of the distances to each of the illumination fibers are the same. Accordingly, because each of the scattering angles of scattered light received by each of the first light-receiving fiber 37, the second light-receiving fiber 38, and the third light-receiving fiber 39 is uniquely fixed, it is possible to receive an angular component, which is the same component as that obtained when using a single illumination fiber, having the light level increased by a factor of 2. Accordingly, light having a sufficient intensity can be irradiated and the diameter of the measurement probe can be reduced.

According to the third embodiment of the present invention described above, in the measurement probe 6, if a combination of light-receiving fiber is taken as a reference with respect to two illumination fibers, the first illumination fiber 35 and the second illumination fiber 36 are arranged at positions where the sums of the distances to each of the illumination fibers are the same. Accordingly, light having a sufficient intensity can be irradiated and the diameter of the measurement probe 6 can be reduced.

Furthermore, according to the third embodiment of the present invention, because each fiber can be arranged two-dimensionally, the measurement probe 6 can be easily produced.

### Fourth Embodiment

In the following, a fourth embodiment of the present invention will be described. With a bio-optical measurement apparatus according to the fourth embodiment, the configuration of a measurement probe differs from that in the embodiments described above. Accordingly, in the following, only the configuration of the measurement probe in the bio-optical measurement apparatus according to the fourth embodiment will be described. Components that are identical to those in embodiments above are assigned the same reference numerals.

FIG. 8 is a sectional view illustrating a distal end surface 70 of a measurement probe 7 in the bio-optical measurement apparatus 1 according to a fourth embodiment of the present invention.

As illustrated in FIG. 8, the measurement probe 7 includes four first illumination fibers 35 and eight first light-receiving fibers 37. In the measurement probe 7, if the center of the measurement probe 7 is taken as a reference, the distances to each of the first illumination fibers 35 are the same. Specifically, each of the first illumination fibers 35 is arranged at a position on a circle with respect to the center of gravity G1 of the measurement probe 7. Furthermore, each of the first light-receiving fibers 37 is also arranged at a position on the circle with respect to the center of gravity G1 of the measurement probe 7.

In this way, in the measurement probe 7, the first illumination fibers 35 and the first light-receiving fibers 37 are arranged at positions on the circle with respect to the center of gravity G1 of the measurement probe 7. Accordingly, because each of the scattering angles of scattered light received by each of the first light-receiving fibers 37 is uniquely fixed, it is possible to receive an angular component, which is the same component as that obtained when using a single illumination fiber, having a light level increased by a factor of 4. Accordingly, light having a sufficient intensity can be irradiated and the diameter of the measurement probe can be reduced.

According to the fourth embodiment of the present invention described above, in the measurement probe 7, the first illumination fibers 35 and the first light-receiving fibers 37 are arranged at positions on a circle with respect to the center of gravity of the measurement probe 7. Accordingly, light having a sufficient intensity can be irradiated and the diameter of the measurement probe can be reduced.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A measurement probe that is detachably connected to a bio-optical measurement apparatus which performs optical measurement on body tissue, the measurement probe comprising:
two illumination fibers configured to irradiate the body tissue with low coherent light having a short spatial coherence length; and
three or more light-receiving fibers configured to receive scattered light at multiple angles, wherein
the three or more light-receiving fibers are arranged on a perpendicular bisector of a line segment connecting centers of the two illumination fibers at a distal end surface of the measurement probe.

2. A bio-optical measurement apparatus that includes the measurement probe according to claim 1.
